(19) United States
(12) Patent Application Publication
(10) Pub. No.: US 2026/0253675 A1
(43) Pub. Date: Aug. 27, 2026

(54) SYSTEM FOR PREDICTING PROTEIN STRUCTURE AND METHOD THEREFOR

(71) Applicant: LG MANAGEMENT DEVELOPMENT INSTITUTE CO., LTD., Seoul (KR)

(72) Inventors: Do Yeong HWANG, Seoul (KR); Ki Young KIM, Seoul (KR); Soo Rin YIM, Seoul (KR); Sung Joon PARK, Seoul (KR); Kyung Wook LEE, Seoul (KR)

(21) Appl. No.: 19/640,281

(22) Filed: Apr. 6, 2026

Related U.S. Application Data
(63) Continuation of application No. PCT/KR2025/022634, filed on Dec. 23, 2025.

(30) Foreign Application Priority Data
Feb. 26, 2025 (KR) 10-2025-0025266
Nov. 3, 2025 (KR) 10-2025-0163418

(51) Int. Cl. G16B 40/00; G06N 20/00; G16B 15/20

(52) U.S. Cl. CPC G16B 40/00; G06N 20/00; G16B 15/20

(57) ABSTRACT

An artificial intelligence system includes at least one memory storing instructions and at least one processor configured to: receive protein amino acid sequence information; generate an embedding of the sequence using at least one AI model; input the embedding into a first AI model and a second AI model to produce respective logit vectors; generate prediction distributions by applying temperature-scaled softmax functions to the logits; compute a soft loss by using the first model's prediction distribution as a soft target and comparing it to the second model's prediction distribution; compute a loss function including the soft loss; and train the second AI model to predict a protein structure from amino acid sequence information by reducing the loss function.

### CROSS REFERENCE TO RELATED APPLICATIONS

This application is a Bypass Continuation of International Patent Application No. PCT/KR2025/022634, filed on Dec. 23, 2025, which claims priority from and the benefit of Korean Patent Application Nos. 10-2025-0025266, filed on Feb. 26, 2025 and 10-2025-0163418, filed on Nov. 3, 2025, which is hereby incorporated by reference for all purposes as if fully set forth herein.

### BACKGROUND

### Field

Embodiments of the invention relate generally to a system for probabilistic circuit-based protein structure prediction and a method therefor capable of improving the accuracy and efficiency of protein structure prediction and performing various probabilistic inferences.

### Discussion of the Background

It has long been known that proteins are core molecules of living organisms that are the source of various life phenomena, but the structure and functions of proteins have not been accurately elucidated until recently. With the completion of the genome project and the dramatic development of gene sequencing technology, a considerable number of amino acid sequences constituting proteins of living organisms have been revealed, but aspects such as the structure, properties, and functions of proteins have still not been sufficiently elucidated.

Recently, artificial intelligence-based protein structure prediction technologies have developed dramatically. In particular, AlphaFold, developed by DeepMind, has achieved considerable prediction accuracy by combining diffusion models and the like. Diffusion models probabilistically model protein folding process, but have the disadvantage of very high computational costs because they must undergo iterative sampling and inference processes. Moreover, generative models such as diffusion models or variational autoencoders are excellent at generating entire structures, but have structural limitations in efficiently handling complex probabilistic queries, such as predicting conditional structures with a portion of a specific amino acid sequence fixed, or inferring structural uncertainty under specific environmental variables.

Therefore, the present inventors aim to provide a system for predicting protein structure and a method therefor that can alleviate the difficulty of the learning process and quickly obtain a high-performance probabilistic circuit model by combining a probabilistic circuit module with a conventional protein structure prediction model using a diffusion model through the present disclosure.

The above information disclosed in this Background section is only for understanding of the background of the inventive concepts, and, therefore, it may contain information that does not constitute prior art.

### SUMMARY

The technical problem to be solved by the present disclosure is to provide a system for predicting protein structure and a method therefor capable of sophisticated structure prediction and uncertainty inference according to partial sequences or environmental conditions, which is difficult to perform with conventional protein structure prediction models.

To solve the above problem, the present disclosure provides an artificial intelligence system and a computer-implemented method.

Additional features of the inventive concepts will be set forth in the description which follows, and in part will be apparent from the description, or may be learned by practice of the inventive concepts.

An artificial intelligence system includes: at least one memory configured to store instructions; and at least one processor configured to execute instructions to:
receive, by at least one artificial intelligence (AI) model stored in the memory, protein amino acid sequence information and generate an embedding for the protein amino acid sequence information; enable a first AI model and a second artificial intelligence model to each generate a logit from the embedding, and each generate a prediction distribution by applying a softmax function using temperature parameters to each generated logit; and enable at least one AI model to obtain a soft loss function by using the prediction distribution generated by the first AI model as a soft target and comparing with the prediction distribution generated by the second AI model, and obtain a loss function using the soft loss function; and train the second AI model to predict a protein structure from protein amino acid sequence information, wherein the training of the second AI is performed such that the loss function is reduced.

In an embodiment, the first AI model may use a diffusion model.

In an embodiment, the second AI model may be a probabilistic circuit model.

In an embodiment, the probabilistic circuit model may be a sum-product network or a probabilistic sentential decision diagram.

In an embodiment, the loss function may be obtained using a soft loss function and a hard loss function, and the hard loss function obtains a difference with a prediction distribution generated through the second AI model using a ground truth label as a hard target.

In an embodiment, in the training of the second AI model such that the loss function may be reduced, the training is performed by additionally using one method selected from expectation maximization or stochastic gradient descent.

In an embodiment, generating a predicted value for the target protein structure from the amino acid sequence information of the target protein by use of the second AI model and generating one or more value selected from the group consisting of confidence, probability distribution, and uncertainty regarding the prediction.

A computer-implemented method includes: receiving, by at least one artificial intelligence (AI) model stored in the memory, protein amino acid sequence information and generating an embedding for the protein amino acid sequence information; enabling a first AI model and a second AI model to each generate a logit from the embedding, and each generate a prediction distribution by applying a softmax function using temperature parameters to each generated logit; enabling at least one AI model to obtain a soft loss function by using the prediction distribution generated by the first AI model as a soft target and comparing with the prediction distribution generated by the second AI model, and obtain a loss function using the soft loss function; and training the second AI model to predict a protein structure from protein amino acid sequence information, wherein the training of the second AI model is performed such that the loss function is reduced.

In another embodiment, the first AI model may use a diffusion model.

In another embodiment, the second AI model may be a probabilistic circuit model.

In another embodiment, the probabilistic circuit model may include a sum-product network or a probabilistic sentential decision diagram.

In another embodiment, the loss function is obtained using a soft loss function and a hard loss function, and the hard loss function obtains a difference with a prediction distribution generated through the second AI model using a ground truth label as a hard target.

In another embodiment, in the training of the second AI model such that the loss function is reduced, the learning is performed by additionally using one method selected from expectation maximization or stochastic gradient descent.

In another embodiment, the second AI model for which training is completed may be used to calculate a predicted value for a structure of a target protein from target protein amino acid sequence information, and to calculate a value for one or more selected from the group consisting of reliability, probability distribution, and uncertainty for the prediction.

In still another embodiment, a program stored on a computer-readable recording medium on a computer for executing a method includes: receiving, by at least one artificial intelligence (AI) model stored in the memory, protein amino acid sequence information and generating an embedding for the protein amino acid sequence information; enabling a first AI model and a second AI model to each generate a logit from the embedding, and each generate a prediction distribution by applying a softmax function using temperature parameters to each generated logit; enabling at least one artificial intelligence model to obtain a soft loss function by using the prediction distribution generated by the first AI model as a soft target and comparing with the prediction distribution generated by the second AI model, and obtain a loss function using the soft loss function; and training the second AI model to predict a protein structure from protein amino acid sequence information, wherein the training of the second AI model is performed such that the loss function is reduced.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a schematic flowchart illustrating the system for predicting protein structure and the method therefor according to one embodiment of the present disclosure.

FIG. 2 is a schematic diagram illustrating the system for predicting protein structure and the method therefor according to one embodiment of the present disclosure.

FIG. 3 is a schematic structural diagram illustrating a probabilistic circuit model used as a second artificial intelligence model according to one embodiment of the present disclosure.

FIG. 4 is a schematic block diagram illustrating an apparatus capable of executing the system for predicting protein structure and the method therefor according to one embodiment of the present disclosure.

FIG. 5 is a schematic diagram illustrating an exemplary on-premise full stack structure.

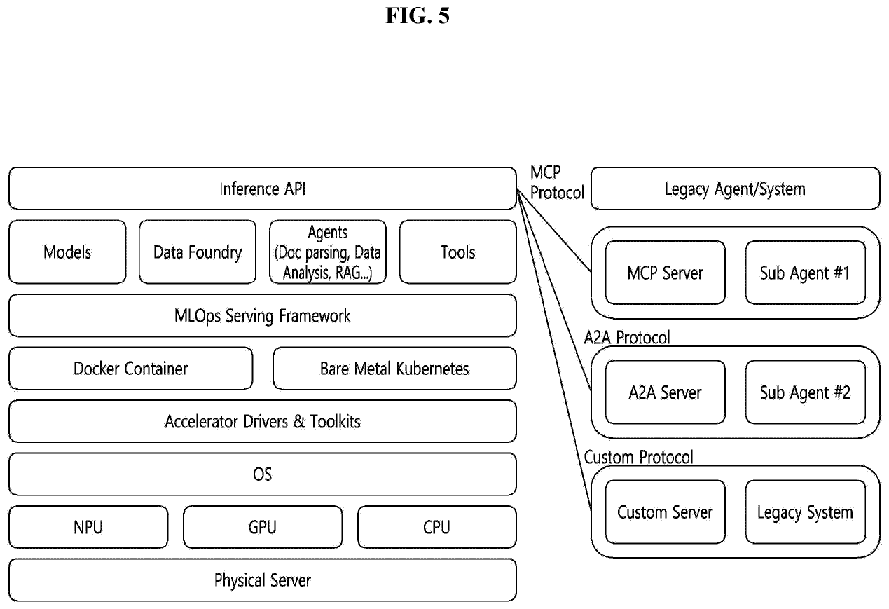

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

The expression “configured to (or set to)” used throughout the present disclosure may be used interchangeably with, for example, “suitable for”, “having the capacity to”, “designed to”, “adapted to”, “made to”, or “capable of”, depending on the situation. The term “configured to (or set to)” may not necessarily mean only “specifically designed to” in terms of hardware. Instead, in some situations, the representation “a system configured to” may mean that the system is “capable of” together with other devices or components. For example, the phrase “a processor configured to (or set to) perform A, B, and C” may refer to a dedicated processor (e.g., an embedded processor) for performing the corresponding operations, or a general-purpose processor (e.g., a CPU or application processor) capable of performing the corresponding operations by executing at least one software program stored in memory.

Functions related to artificial intelligence according to the present disclosure are operated through a processor and a memory. The processor may include one or more processors. In this case, the one or more processors may be a general-purpose processor such as a CPU, application processor (AP), a digital signal processor (DSP), and the like, a graphics-dedicated processor such as a graphics processing unit (GPU), a vision processing unit (VPU), or an artificial intelligence-dedicated processor such as a neural processing unit (NPU). The one or more processors control processing of input data according to predefined operation rules or artificial intelligence models stored in the memory. Alternatively, when the one or more processors are artificial intelligence-dedicated processors, the artificial intelligence-dedicated processors may be designed with a hardware structure specialized for processing a specific artificial intelligence model.

Artificial intelligence may be a field of computer engineering and information technology that studies methods to enable computers to perform tasks that are performed by human intelligence such as thinking, learning, and self-development, and this means enabling computers to imitate human intelligent behavior. Furthermore, artificial intelligence may not be present by itself, but is directly and indirectly related to other fields of computer science. Especially in modern times, attempts are very actively made to introduce artificial intelligence elements in various fields of information technology to utilize them for solving problems in those fields.

Machine learning may be considered a branch of artificial intelligence that is a research field for giving computers the ability to learn without explicit programming. More specifically, machine learning may refer to technologies for developing systems and algorithms that learn from empirical data, generate predictions, and improve their performance over time. Rather than executing strictly predefined static program instructions, machine learning algorithms take an approach of building a specific model to derive predictions or decisions based on input data, rather than executing strictly defined static program instructions.

Many machine learning algorithms may be developed regarding how to classify data in machine learning. Representative examples of the machine learning algorithms may include Decision Tree, Bayesian network, support vector machine (SVM), and Artificial Neural Network (ANN), and the like.

A Decision Tree may be an analytical method that plots decision rule into a tree-like structure to perform classification and prediction. A Bayesian network may be a model that represents probabilistic relationships (conditional independence) between multiple variables in a graph structure. Bayesian network may be suitable for data mining through unsupervised learning.

A support vector machine may be a model of supervised learning for pattern recognition and data analysis, mainly used for classification and regression analysis. An Artificial Neural Network may be a model of the working principles of biological neurons and the connections between neurons, and is an information processing system in which multiple neurons called nodes or processing elements are connected in the form of a layered structure.

An Artificial Neural Network may be a model used in machine learning, and may be a statistical learning algorithm inspired by biological neural networks (especially the brain of the animal central nervous system) in machine learning and cognitive science. Specifically, an Artificial Neural Network may refer to all models in which artificial neurons (nodes) formed by the connections of synapses form a network, which, through learning, changes the strength of the connections of the synapses and obtains problem-solving capability. The term ‘Artificial Neural Network’ may be used interchangeably with the term ‘Neural Network.’

An Artificial Neural Network may include multiple layers, each of which includes a plurality of neurons. In addition, an Artificial Neural Network may include synapses that connect neurons to neurons. An Artificial Neural Network may generally be defined by the following three factors: connection patterns between neurons in different layers, a learning process that updates the weights of the connections, and an activation function that generates output values from the weighted sum of inputs received from a previous layer.

Artificial Neural Networks may include network models such as Deep Neural Network (DNN), Recurrent Neural Network (RNN), Bidirectional Recurrent Deep Neural Network (BRDNN), Multilayer Perceptron (MLP), and Convolutional Neural Network (CNN), but are not limited thereto. The term ‘layer’ as used herein may be used interchangeably with the term ‘tier.’

Artificial Neural Networks may be classified into single-layer neural networks and multi-layer neural networks, depending on the number of layers. A typical single-layer neural network may include an input layer and an output layer. In addition, a typical multi-layer neural network may include an input layer, at least one hidden layer, and an output layer.

The input layer may be a layer that receives external data, and the number of neurons in the input layer may be equal to the number of input variables. The hidden layer may be positioned between the input layer and the output layer and receives signals from the input layer, extracts features, and transmits the features to the output layer. The output layer receives signals from the hidden layer and outputs output values based on the received signals. Input signals between neurons are multiplied by respective connection strengths (weights) and then summed, and when this sum may be greater than a threshold value of the neuron, the neuron may be activated and outputs an output value obtained through an activation function.

Meanwhile, a deep neural network including a plurality of hidden layers between the input layer and the output layer may be a representative Artificial Neural Network that implements deep learning, which is a type of machine learning technology.

A machine learning workflow includes a series of processes for collecting data for learning and validation and training a model after modeling, and may include processes of training data collection, data inspection and exploration, data preprocessing and cleaning, and modeling and training.

The present disclosure provides a system and method for rapidly and efficiently predicting protein structure as follows.

### 1. Protein Data Acquisition

The protein data that may be used for the system for predicting protein structure and the method therefor of the present disclosure includes, without limitation, any data that may be utilized in protein structure prediction. For example, protein amino acid sequence information, multiple sequence alignment information, contact maps, secondary structure prediction information, and the like may be used. According to one embodiment of the present disclosure, the protein data includes at least one protein amino acid sequence information.

### 2. Training Data Inspection and Exploration

Once training data for learning of an artificial model is collected, the collected training data may be subjected to inspection and exploration for data structure, noise data, and data cleaning methods for machine learning application.

This data inspection and exploration step may be referred to as an exploratory data analysis (EDA) step, and EDA may be said to be a process of observing and understanding collected data from various perspectives. Before learning the data, independent variables, dependent variables, variable types, data types of variables, and the like may be inspected through visualization such as graphs and statistical tests, and features of the data and inherent structural relationships may be confirmed in advance. Through such EDA, by examining distribution and values of the data, a phenomenon represented by the data may be better understood, and potential problems with the data may be discovered. Furthermore, through a process of inspecting the data from various perspectives, various patterns that could not have been generated in a problem definition step may be discovered, and based on this, existing hypotheses may be modified or new hypotheses may be established. The exploratory data analysis may largely include a process of exploring outliers of the data and a process of analyzing relationships between data attributes.

The process of exploring for outliers involves checking for the presence of outliers in the data, and may include sampling methods, statistical methods, and visualization methods. The sampling method involves extracting random samples from the data to determine the overall trend and anomalies of the data values. The statistical method may use summary statistics such as mean, median, mode for determining the center of the data, or range and variance for determining the dispersion of the data. The visualization method may use probability density functions, histograms, dot plots, word clouds, time series charts, maps, and the like to determine which statistical indicators may be appropriate for the individual attributes of the collected data. However, when using statistical indicators, it should be noted that the mean reflects all data values in the set and is therefore affected by outliers, while the median uses only a single value positioned in the middle and may provide representative results even in the presence of outliers.

The process of analyzing relationships between data attributes involves finding combinations of attributes within the data that have meaningful correlations with each other. The relationship analysis may be performed differently depending on the combination of attributes between categorical variables (qualitative), which may not be represented numerically but may be arbitrarily quantified, and numeric variables (quantitative), which may be quantified. A categorical-categorical relationship may provide the number of values corresponding to each pair of attribute values using cross tables and mosaic plots. A numeric-categorical relationship may be obtained by observing statistical values by category (mean, median, etc.) or be visually represented using box plots. A numeric-numeric relationship may analyze the association between two attributes using correlation coefficients. A correlation coefficient of −1 indicates a negative correlation where the two attributes change in opposite directions, 0 indicates no correlation, and 1 indicates a positive correlation where the two attributes always change in the same direction. The relationship between two attributes having a correlation coefficient may show various patterns, which may be visually represented using scatter plots.

### 3. Pre-Processing of Training Data

Data that has completed inspection and exploration undergoes data preprocessing to process it into a form suitable for models for machine learning. Data preprocessing involves purifying data and converting it into a form that the model may understand. Data preprocessing generally may include handling missing data, outlier removal, data scaling, categorical data encoding, feature selection and extraction, and data transformation. All or some of the detailed processes of data preprocessing may be selectively performed, and a separate machine learning model may be used for data preprocessing.

Handling missing Data may be a process of handling missing values that may be present in data, and the missing values may be displayed as NaN (Not a Number) or empty values or may be deleted. As the missing values may be filled or deleted within the data, integrity of the data may be improved, and when filling the missing values, an average value, a median value, a mode value, or the like may be used.

Outlier removal may be removing outliers, which may be values that deviate from general data patterns in data. Since the outliers may degrade performance of a model, they must be removed or replaced, and the outliers may be identified and corresponding rows or columns may be deleted or replaced with other values.

Data scaling may be a process of adjusting a size of data, and through the data scaling, a range of the data may be adjusted and performance of a model may be improved or a convergence speed may be improved. Through the data scaling, features of the data may be adjusted to a similar range, and generally, standardization and normalization may be applied to the data scaling.

Categorical data encoding may be converting a categorical variable represented by a string or integer value that may not be directly input to a model into a numeric form that may be input to the model. Generally, one-hot encoding or label encoding may be used to convert a categorical variable into a numeric form.

Feature selection and extraction may be for selecting the most useful features for model learning or extracting new features to enhance the performance of the model, and through this process, the complexity of the model may be reduced and overfitting may be prevented.

Data transformation may be for transforming data to extract new information or to enable the model to better understand the data, and may include tokenization of text data or preprocessing of image data. Through data transformation, useful features may be extracted from original data or the data may be transformed into an appropriate form, thereby enhancing the performance of the model.

The above-described data preprocessing may achieve the effect of enhancing the performance of the machine learning model and ensuring stability.

Meanwhile, when the collected data may not be preprocessed as needed, tokenization, cleaning and normalization may be carried out according to the intended use of the data.

In order for text to be understood and processed by computers, the text must be properly converted into numbers. Since the performance of natural language processing varies significantly depending on how words may be represented, many techniques have been proposed to quantify words. Word embedding, which converts each word into a vector using ANN learning, may be now frequently used.

### 4. Structure of Method for Predicting Protein Structure

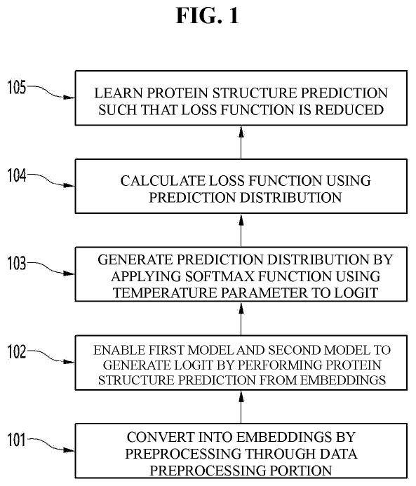

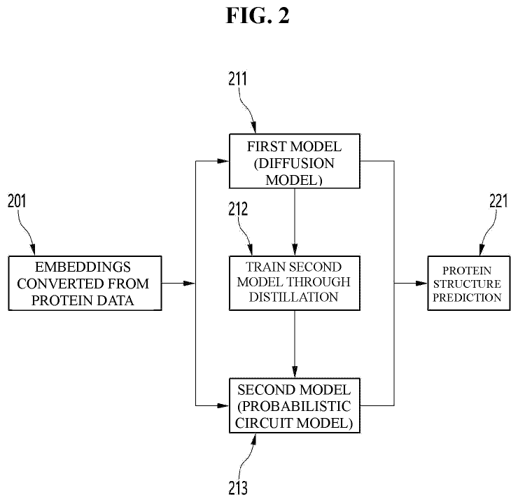

The present disclosure relates to a system for predicting protein structure and a method therefor, which in one embodiment may operate in a manner as shown in FIGS. 1 and 2.

Data such as protein amino acid sequences and/or multiple sequence alignment information input to the system of the present disclosure may be preprocessed through a data preprocessing portion and converted into embeddings 101.

A first artificial intelligence model of the present disclosure performs protein structure prediction from the embeddings to generate logits 102. The first artificial intelligence model may be a large ANN that has been pre-trained and shows high performance in protein structure prediction. The ANNs that may be used as the first artificial intelligence model in the present disclosure may not be particularly limited, but a representative example may be a diffusion model of AlphaFold 3, which generates three-dimensional structure prediction values of proteins and probability distributions of the structures based on input embeddings. The system and method of the present disclosure may also be applied to other models introduced after the date of filing the present disclosure.

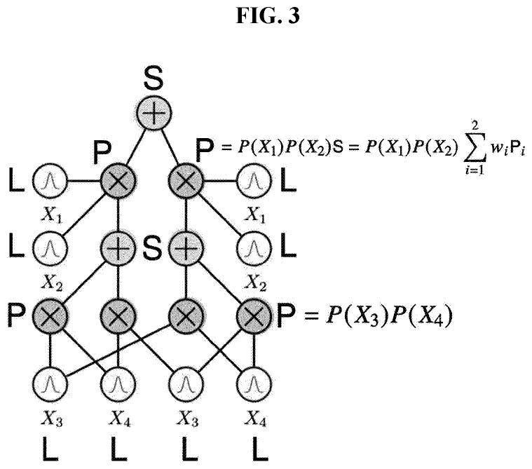

In the system and method of the present disclosure, a second artificial intelligence model performs learning by mimicking the first artificial intelligence model. The second artificial intelligence model may be a probabilistic circuit model as shown in FIG. 3. The probabilistic circuit models that may be used in the present disclosure are not particularly limited, but may be implemented as a sum-product network (SPN) or a probabilistic sentential decision diagram (PSDD), and the like. The second artificial intelligence model generates logits, which are unnormalized prediction score vectors, from the embeddings generated by preprocessing protein data 102.

In the present disclosure, the learning portion performs learning so that the protein structure prediction of the second artificial intelligence model mimics the protein structure prediction of the first artificial intelligence model as much as possible. The learning portion of the present disclosure generates a prediction distribution by applying a softmax function using a temperature parameter to logits output by each of the first artificial intelligence model and the second artificial intelligence model 103.

In the present disclosure, a soft target refers to a probability distribution obtained by passing logits, which are output values of an artificial intelligence model, through a softmax function to which a temperature parameter (T) may be applied. This may be used for learning through knowledge distillation and plays an important role in transferring knowledge of the first artificial intelligence model to the second artificial intelligence model in the present disclosure. According to one embodiment of the present disclosure, the prediction distribution may be obtained through a function represented by Equation (1) below.

$$Q ( i ) - \frac{exp ( {z}_{i} / T )}{{\sum }_{j \text{?} 1}^{K} exp ( {z}_{j} / T )} \qquad [ Equation ( 1 ) ]$$

$$\text{?} \text{indicates text missing or illegible when filed}$$

In Equation (1) above, Q(i) is a probability value for an i-th class of a prediction distribution output by a model, zᵢ is a value corresponding to the i-th class in a logit vector Z of the model, T is a temperature parameter, and K is a total number of classes to be classified by the model.

The learning portion sets a prediction distribution generated through the first artificial intelligence model as a soft target, and obtains a soft loss function by comparing with a prediction distribution generated through the second artificial intelligence model 104. The soft loss function, which is a calculation of the difference between the prediction distribution of the first artificial intelligence model and the prediction distribution of the second artificial intelligence model, represents the similarity of the prediction distributions generated by the two models. The loss functions used in the system and method of the present disclosure are not particularly limited, but the difference between the prediction distributions of the first artificial intelligence model and the second artificial intelligence model may be minimized by using loss functions such as, for example, mean square error, Kullback-Leibler divergence.

In the present disclosure, a loss function may be additionally used to further improve protein structure prediction learning performance. The loss function additionally used in the present disclosure may be a loss function obtained using a hard target which may be a ground truth label in one-hot encoding form (i.e., a hard loss function). The hard loss function may be a calculation of the difference between the prediction of the second artificial intelligence model and an actual ground truth label. In the present disclosure, the soft loss function and the hard loss function may be used in combination, and for example, learning may be performed by setting a value obtained by adding the soft loss function and the hard loss function with respective weights as a final loss function.

In the present disclosure, in order to minimize the loss function, parameters at each node (sum node, product node, etc.) of the probabilistic circuit may be updated using an optimization algorithm such as expectation maximization or stochastic gradient descent.

The system and method of the present disclosure performs learning such that the loss function may be reduced 105, and through the above-described process, the second artificial intelligence model can effectively simulate the probability distribution predicted by the first artificial intelligence model.

The second artificial intelligence model, which has completed learning according to the system and method of the present disclosure, may be lightweight and efficient and performs inference and prediction of protein structure with high performance compared to the first artificial intelligence model.

After completing learning, the second artificial intelligence model can flexibly perform various prediction and inference tasks such as conditional probability (e.g., predicting the entire protein structure when a protein amino acid sequence of a certain part may be fixed), marginalization (e.g., predicting protein structure when some protein amino acid sequences may be uncertain), sampling (e.g., simulation of a new protein structure), and can easily calculate uncertainty information such as prediction reliability, probability distribution, variance, and likelihood using the structural properties of the probabilistic circuit. Furthermore, the probability distribution and protein structure can be predicted and uncertainty result values can be calculated immediately with a single or a small number of operations without the repetitive sampling process of the diffusion module, which is the first artificial intelligence model.

The second artificial intelligence model that has completed learning may be used in parallel or sequentially with the first artificial intelligence model, and may further improve the reliability of the final structure prediction by utilizing ensemble techniques, and the like.

The system and method of the present invention may increase the degree of freedom of probabilistic inference. While conventional diffusion models or variational autoencoder models, or the like have difficulty performing complex conditional and marginalization operations, the system and method of the present disclosure enable immediate and intuitive inference. For example, it may be possible to assume mutations or evaluate the structural stability of a protein for specific environmental conditions such as pH by marginalization.

The system and method of the present invention enable efficient learning and inference. By transferring knowledge already learned in the first artificial intelligence model to the second artificial intelligence model, the difficult initial learning problem of the circuit module can be alleviated. Through this, protein structure prediction and uncertainty analysis can be performed with a single or a small number of operations of the circuit without the burden of repeated sampling of the diffusion model.

The system and method of the present invention enable precise uncertainty estimation. By transferring knowledge already learned in the first artificial intelligence model to the second artificial intelligence model, the difficult initial learning problem of the circuit module can be alleviated. Thus, protein structure prediction and uncertainty analysis can be performed with a single or a small number of operations of the circuit without the burden of repeated sampling of the diffusion model.

The system and method of the present invention have an advantage in calculating structural reliability, which has critical significance in the development of new drugs.

The system of the present invention has high utility in various application fields, including new drug development and life science research, through this inference capability.

### Mixture of Experts (MoE) Architecture

In one embodiment of the present disclosure, a system for predicting a phenotype may be performed by utilizing a model architecture such as MoE. In this case, MoE may refer to an architecture of a machine learning model that combines multiple expert models to solve complex problems.

Such MoE may include expert models, which are multiple small networks designed to learn different parts and/or different features of predetermined data and perform data processing operations accordingly, and a gating network that evaluates the performance of each expert model and determines based thereon which expert model is most suitable for assigning a specific task according to the predetermined data.

Therefore, according to the MoE architecture, a gating network that acquires predetermined input data determines probabilistic or deterministic task assignment for each expert model, and the selected expert models may perform data processing for a specific task by performing their respective tasks and returning the results thereof.

The MoE model used according to one embodiment of the present disclosure may refer to a predetermined MoE model implemented according to a universal method known in the relevant technical field. For example, the MoE model may include a Switch Transformer, Conditional Computation in Neural Networks, Sparse Mixture of Experts, and/or Megatron-LM, and the like.

In addition, in one embodiment of the present disclosure, the MoE model, based on a combination of a plurality of Specialized Models (SM) and a Router (Gating Network, RT) may be included, and a domain-specific specialized model-based MoE model may also be included.

By utilizing such MoE, when handling complex tasks or large data sets, or the like, the overall efficiency and performance of the system for predicting the phenotype of the present disclosure may be enhanced by concentrating computational resources by activating only specific parts.

### 5. Computing System Device

An embodiment of the present invention may be implemented as an Application Specific Integrated Circuit (ASIC) that is manufactured to suit specific application fields and special functions of devices.

ASICs may be also referred to as custom semiconductors, and unlike standard semiconductors that have established specifications and can be applied to any electronic product or application as long as certain requirements are met, custom semiconductors may be used for a specific product or function and may be integrated circuits that semiconductor manufacturers fabricate according to specific orders. In other words, custom semiconductors may be designed and manufactured to perform only the functions necessary for a specific device or specific function, and custom semiconductors may be broadly classified according to design method into full custom ICs, which are designed and manufactured from the beginning according to user requirements, and semicustom ICs, which are designed and manufactured using some standardized designs.

Custom semiconductors may be mainly used in communication systems, high-performance computing systems, consumer electronics, automobiles, industrial automation, medical devices, military, aerospace industry, and the like, and have recently been applied to artificial intelligence (AI) semiconductors that execute large-scale computations required for artificial intelligence (AI) implementation with high performance and power efficiency.

Custom semiconductors may be used as core components of network routers, switches, modems, and the like in communication systems to perform data packet processing, protocol conversion, signal processing, and the like, providing high throughput and low latency. In high-performance computing systems, ASICs may be used as core components for high-speed processing and parallel processing. In consumer electronics, such as digital cameras, smartphones, tablets, and game consoles, ASICs provide high-performance and low-power solutions required to perform specific functions. In the automotive industry, ASICs may be used to control various electronic systems inside vehicles, and in industrial automation systems, ASICs provide solutions for high-precision control and high-performance processing.

The ASIC to which an embodiment of the present invention is applied includes a memory in which a discrete memory interface (I/F) is implemented, and may include a plurality of functional blocks requesting memory access. Each functional block may be a direct memory access (DMA) functional block, a processor, a video processor, a cache controller, a decompression block, or a data path block. The basic configuration of the ASIC may include: transistors that amplify or switch electrical signals; logic gates, which may be circuits that combine transistors to perform logical functions; memory cells that store data; analog circuits, which may be circuits that combine transistors to process continuous voltage or current; and intellectual property (IP) cores, such as microprocessors, digital signal processors (DSPs), and graphics cores, which may be pre-designed to perform specific functions.

The ASIC may also include a discrete memory I/F that interfaces with a discrete memory and an embedded memory I/F that interfaces with an embedded memory. The discrete memory I/F may be connected to each functional block to receive memory access signals (e.g., control signals, address signals, and data signals) and, based on these input signals, generate signals that control the discrete memory. The embedded memory I/F may be connected to each functional block to receive memory access signals (e.g., control signals, address signals, and data signals) and, based on these input signals, generate modified memory access signals that control the embedded memory. The discrete memory I/F and the embedded memory I/F may be designed within a memory control block of the ASIC to provide a memory control structure that can be flexibly applied to both the discrete memory and the embedded memory.

Furthermore, an ASIC for an ANN may includes multiple neurons arranged in an array and many synaptic circuits. Each neuron may include a register, a microprocessor, and at least one input, and each synaptic circuit may include a memory for storing synaptic weights. In this case, each neuron in the ASIC may be connected to at least one other neuron through one of the many synaptic circuits.

Although the present disclosure has been described above as being generally implementable by a computing device, those skilled person in the art will understand that the present disclosure may be implemented in combination with computer-executable instructions and/or other program modules executable on at least one computer and/or as a combination of hardware and software.

Those of ordinary skill in the art of the present disclosure will understand that information and signals may be represented using any of a variety of different technologies and techniques. For example, data, instructions, commands, information, signals, bits, symbols, and chips that may be referenced in the above description may be represented by voltages, currents, electromagnetic waves, magnetic fields or particles, optical fields or particles, or any combinations thereof.

Those of ordinary skill in the art of the present disclosure will understand that the various exemplary logical blocks, modules, processors, means, circuits, and algorithmic steps described in connection with the embodiments disclosed herein may be implemented by electronic hardware, various forms of program or design code (herein referred to as software for convenience), or a combination of both. To clearly describe this interchangeability of hardware and software, various exemplary components, blocks, modules, circuits, and steps have been generally described above in relation to their functions. Whether such functions are implemented as hardware or software depends upon the particular application and design constraints imposed on the overall system. Those of ordinary skill in the art of the present disclosure may implement the described functions in varying ways for each particular application, but such implementation decisions should not be interpreted as departing from the scope of the present disclosure.

The various embodiments presented herein may be implemented as a method, apparatus, or article manufactured using standard programming and/or engineering techniques. The term “article” encompasses a computer program, carrier, or media accessible from any computer-readable storage device. For example, computer-readable storage media include magnetic storage devices (e.g., hard disk, floppy disk, magnetic strips, etc.), optical disks (e.g., CD, DVD, etc.), smart cards, and flash memory devices (e.g., electrically erasable programmable read-only memory (EEPROM), card, stick, key drive, etc.), but are not limited thereto. In addition, the various storage media presented herein include at least one device and/or other machine-readable media for storing information.

It may be to be understood that the specific order or hierarchy of steps in the processes presented is an example of exemplary approaches. It may be to be understood that based on design priorities, the specific order or hierarchy of steps in the processes may be rearranged within the scope of the present disclosure. The accompanying method claims present elements of the various steps in a sample order but are not meant to be limited to the specific order or hierarchy presented.

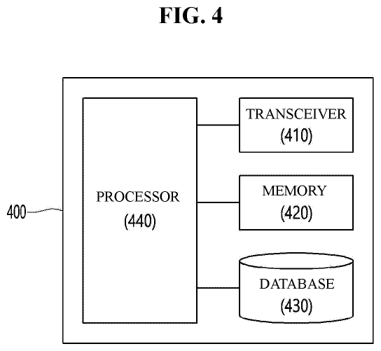

FIG. 4 shows an example of a block diagram illustrating a computing system apparatus 400 of the present disclosure.

The system and method of the present disclosure may be performed by any suitable means capable of performing the corresponding functions. Such means may include various hardware, software components, modules, and combinations thereof, including circuits, processors, ASICs, and field programmable gate arrays (FPGAs) but not limited thereto.

Referring to FIG. 4, a computing system 400 implementing the ANN of the present disclosure may include a transceiver 410, a memory 420, a database 430, and a processor 440. However, not all of the components shown in FIG. 4 are essential components of the computing system 400. The computing system 400 may be implemented by more components than those shown in FIG. 4, and the computing system 400 may be implemented by fewer components than those shown in FIG. 4. In addition, the transceiver 410, the memory 420, and the processor 440 may be implemented in the form of a single chip.

In one embodiment, the transceiver 410 may communicate with a terminal connected to the computing system device 400 in a wired or wireless manner, or with another electronic device.

Various types of data such as programs and files, for example, applications may be installed and stored in the memory 420. The processor 440 may access and use data stored in the memory 420 or store new data in the memory 420. In addition, the memory 420 may store at least one instruction. The processor 440 may execute at least one instruction stored in the memory.

The processor 440 controls the overall operation of the computing system apparatus 400, wherein the processor 440 may include at least one or a plurality of electrically connected processors, such as a CPU, a GPU, an ASIC, a DSP, a digital signal processing device (DSPD), a programmable logic device (PLD), a Field Programmable Gate Array, a controller, a micro-controller, a microprocessor, and/or an electrical unit for performing other functions.

The processor 440 may control other components included in the computing system device 400 to perform operations for operating the computing system device 400.

The database 430 may store various learning data for training a learning model. In addition, the database 430 may store protein amino acid sequence information, protein structure information, simulation result information, and the like, and, in various embodiments, the database may store output data outputted by the learning model. In FIG. 4, the computing system apparatus 400 may be illustrated to include the database 430, but the database 430 may be provided outside the device. In this case, the database 430 may be connected to the computing system apparatus 400 in a wired or wireless manner.

Furthermore, the learning model of the present disclosure may be implemented outside the computing system apparatus 400 (e.g., implemented in a cloud-based manner) or may be included within the computing system apparatus 400.

One embodiment of the present disclosure may also be implemented in the form of a recording medium including instructions that may be executed by a computer, such as program modules executed by a computer. Computer-readable media may be any available media that may be accessed by a computer and include both volatile and non-volatile media, removable and non-removable media. In addition, computer-readable media may include both computer storage media and communication media. Computer storage media include both volatile and non-volatile media and removable and non-removable media implemented in any method or technology for storage of information such as computer-readable instructions, data structures, program modules, or other data. Communication media typically include computer-readable instructions, data structures, or program modules and include any information delivery media.

Throughout the present disclosure, the apparatus may include servers, smartphones, tablet PCs, PCs, TVs, smart TVs, mobile phones, personal digital assistants (PDAs), speakers, laptops, media players, micro servers, e-book object recognition devices, digital broadcasting object recognition devices, kiosks, MP3 players, digital cameras, robot cleaners, home appliances, other mobile or non-mobile computing devices, watches, glasses, hair bands, and rings equipped with communication functions and data processing functions, but is not limited thereto.

### 6. Applications of System for Predicting Protein Structure and Method Therefor

The system for predicting protein structure and the method therefor of the present disclosure can be applied to the prediction of complex structures and interactions of various biomolecules including proteins, DNA, RNA, and small molecules, which can be effectively utilized in medical, pharmaceutical, and life science industries.

Specifically, the system for predicting protein structure and the method therefor of the present disclosure can be used for new drug development and drug design. The system for predicting protein structure and the method therefor of the present disclosure can dramatically reduce development time and cost by accurately predicting how a new drug candidate substance binds to and affects a specific protein, thereby rapidly identifying an effective new drug substance and reducing unnecessary experimentation. Furthermore, the system may be also utilized to predict in advance the possibility that a drug will react with other unintended molecules, thereby reducing potential side effects. This contributes not only to enhancing drug efficacy but also to strengthening drug safety.

The system for predicting protein structure and the method therefor of the present disclosure can also be effectively utilized in industries in the fields of disease research and precision medicine. For example, by predicting how genetic variations cause changes in the structure and function of proteins, the system and method can be used to elucidate fundamental mechanisms of genetic diseases whose causes were previously difficult to determine. Through this, a foundation for precision medicine can be established to design personalized treatments tailored to the characteristics of individual patients. Furthermore, by accurately predicting the binding structures of immune-related molecules such as antigen-antibody complexes during the development of immunotherapeutics, the system and method can contribute to the development of new vaccines or therapeutics.

The system for predicting protein structure and the method therefore of the present disclosure can also be effectively utilized in industrial fields related to protein engineering and enzymes, and can be utilized to design new proteins having specific functions or to optimize the functions of existing proteins. For example, the system and method can play an important role in creating highly efficient enzymes required for industrial processes or in developing disease-resistant crops by analyzing interactions between plants and pathogens in the agricultural field.

By using the system for predicting protein structure and the method therefor of the present disclosure, learning difficulty for large-scale protein data sets can be alleviated compared to the prior art.

In addition, using the system for predicting protein structure and the method therefor of the present disclosure enables various probability queries that are difficult to implement in prior art such as diffusion models, and allows immediate and flexible prediction and inference tasks such as conditional probability, marginalization, and sampling to be performed.

Furthermore, using the system for predicting protein structure and the method therefor of the present disclosure, rapid and efficient protein structure prediction and inference can be performed with only a single or few operations without an iterative sampling process of diffusion models.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. An artificial intelligence system, comprising:
at least one memory configured to store instructions; and
at least one processor configured to execute the instructions to:
receive, by at least one artificial intelligence (AI) model stored in the memory, protein amino acid sequence information and generate an embedding for the protein amino acid sequence information;
enable a first AI model and a second AI model to each generate a logit from the embedding, and each generate a prediction distribution by applying a softmax function using temperature parameters to the each generated logit; and
enable at least one AI model to obtain a soft loss function by using the prediction distribution generated by the first AI model as a soft target and comparing with the prediction distribution generated by the second AI model, and obtain a loss function using the soft loss function; and
training the second AI model to predict a protein structure from protein amino acid sequence information, wherein the training of the second AI model is performed such that the loss function is reduced.

2. The artificial intelligence system of claim 1, wherein the first AI model uses a diffusion model.

3. The artificial intelligence system of claim 1, wherein the second AI model is a probabilistic circuit model.

4. The artificial intelligence system of claim 3, wherein the probabilistic circuit model is a sum-product network or a probabilistic sentential decision diagram.

5. The artificial intelligence system of claim 1, wherein the loss function is obtained using a soft loss function and a hard loss function, and the hard loss function obtains a difference with a prediction distribution generated through the second AI model using a ground truth label as a hard target.

6. The artificial intelligence system of claim 1, wherein in the training of the second AI model such that the loss function is reduced, the training is performed by additionally using one method selected from expectation maximization or stochastic gradient descent.

7. The artificial intelligence system of claim 1, generating a predicted value for the target protein structure from the amino acid sequence information of the target protein by use of the second AI model and generating one or more value selected from the group consisting of confidence, probability distribution, and uncertainty regarding the prediction.

8. A computer-implemented method, comprising:
receiving, by at least one artificial intelligence (AI) model stored in the memory, protein amino acid sequence information and generating an embedding for the protein amino acid sequence information;
enabling a first AI model and a second AI model to each generate a logit from the embedding, and each generate a prediction distribution by applying a softmax function using temperature parameters to each generated logit;
enabling at least one artificial intelligence model to obtain a soft loss function by using the prediction distribution generated by the first AI model as a soft target and comparing with the prediction distribution generated by the second AI model, and obtain a loss function using the soft loss function; and
training the second AI model to predict a protein structure from protein amino acid sequence information, wherein the training of the second AI model is performed such that the loss function is reduced.

9. The method of claim 8, wherein the first AI model uses a diffusion model.

10. The method of claim 8, wherein the second AI model is a probabilistic circuit model.

11. The method of claim 10, wherein the probabilistic circuit model includes a sum-product network or a probabilistic sentential decision diagram.

12. The method of claim 8, wherein the loss function is obtained using a soft loss function and a hard loss function, and the hard loss function obtains a difference with a prediction distribution generated through the second AI model using a ground truth label as a hard target.

13. The method of claim 8, wherein the in the training of the second AI model such that the loss function is reduced, the training is performed by additionally using one method selected from expectation maximization or stochastic gradient descent.

14. The method of claim 8, generating a predicted value for the target protein structure from the amino acid sequence information of the target protein by use of the second AI model and generating one or more value selected from the group consisting of confidence, probability distribution, and uncertainty regarding the prediction.

15. A program stored on a computer-readable recording medium on a computer for executing a method, the method comprising:
receiving, by at least one artificial intelligence (AI) model stored in the memory, protein amino acid sequence information and generating an embedding for the protein amino acid sequence information;
enabling a first AI model and a second AI model to each generate a logit from the embedding, and each generate a prediction distribution by applying a softmax function using temperature parameters to each generated logit;
enabling at least one artificial intelligence model to obtain a soft loss function by using the prediction distribution generated by the first AI model as a soft target and comparing with the prediction distribution generated by the second AI model, and obtain a loss function using the soft loss function; and
training the second AI model to predict a protein structure from protein amino acid sequence information, wherein the training of the second AI model is performed such that the loss function is reduced.
